(19) United States
(12) Patent Application Publication
(10) Pub. No.: US 2025/0009260 A1
(43) Pub. Date: Jan. 9, 2025

(54) METHODS FOR ASSESSMENT, PROGRESS TRACKING AND CHALLENGE POINT TRAINING OF BIOMECHANIC CHARACTERISTICS FOR USERS WEARING ASSISTIVE DEVICES

(71) Applicant: K!M ehf, Kópavogur (IS)

(72) Inventor: Kim De Roy, Kópavogur (IS)

(21) Appl. No.: 18/764,826

(22) Filed: Jul. 5, 2024

Related U.S. Application Data
(60) Provisional application No. 63/524,967, filed on Jul. 5, 2023.

(51) Int. Cl. A61B 5/11; A61B 5/00

(52) U.S. Cl. CPC A61B 5/112; A61B 5/1126; A61B 5/742; A61B 5/7475; A61B 2562/0219

(57) ABSTRACT

Methods and apparatus for assessment, progress tracking and challenge point training of biomechanic characteristics for users wearing assistive devices. In one embodiment, a method of using an IMU sensor for collection of biomechanical data includes collecting data from the IMU sensor during a prescribed exercise being performed by a user; processing the data from the IMU sensor using a machine learning or artificial intelligence engine to determine a type of an assistive device for use with the user; collecting additional data from the IMU sensor when the user is using the assistive device; processing the additional data using the machine learning or artificial intelligence engine to determine an exercise to be performed by the user when using the assistive device; and displaying the additional data on a GUI along with previously collected data for another performance of the exercise. Systems that include IMU sensors and computer-readable media are also disclosed.

### CROSS-REFERENCE TO RELATED APPLICATIONS

This application claims the benefit of priority to U.S. Provisional Patent Application Ser. No. 63/524,967 filed Jul. 5, 2023, entitled “Methods and Apparatus for Assessment, Progress Tracking and Challenge Point Training of Biomechanic Characteristics for Users Wearing Assistive Devices”, the contents of which being incorporated herein by reference in its entirety.

### TECHNOLOGICAL FIELD

The present disclosure relates generally to the field of assessment and enhancement of human performance during use of assistive devices and/or supporting gear, and more particularly in one exemplary aspect to methods, systems, and computer-readable media for the assessment of various biomechanical parameters to quantify and optimize the efficiency of users wearing the aforementioned assistive devices and/or supporting gear.

### BACKGROUND

The utilization of lighter weight and more energy efficient assistive devices and/or supporting gear has grown for over the past two decades. More recently, manufacturers have claimed increased efficiency with the use of certain prosthetic and orthotic devices as compared with their competitors, as do the providers of performance optimizing gear for use with these prosthetic and orthotic devices. These claims being made are often based on research in a controlled environment and/or based on laboratory bench-testing of these products. These claims as to device effectiveness often generally relate to known parameters such as, for example, energy return, flexibility, stability, movement control, as well as symmetry in gait. However, these parameters are often not controlled or assessed while the user of the assistive device and/or supporting gear functions outside of these controlled environments. Therefore, use of these devices outside of these controlled environments often leads to suboptimal and inefficient use of the device. Accordingly, tools are needed which allow users of these devices to track their personal performance on, for example, a day-to-day basis and provide feedback and training/exercise recommendations that are required to maintain or improve certain capability of performance levels while using an assistive device and/or supporting gear. Additionally, such tools would beneficially track user compliance with regards to prescribed exercise and therapies.

### SUMMARY

The present disclosure satisfies the foregoing needs by providing, inter alia, methods and apparatus for the assessment of the effectiveness and efficiency of human-device interaction when a user wears an assistive device and/or supporting gear.

When using an assistive device and/or supporting gear, it is important to keep individual characteristics in mind which might affect the ability for the user to use such a device. The present disclosure objectively measures the effect of the combination of all the influencing parameters for device effectiveness that are translated into functional outcome measures as the user uses the assistive device and/or supporting gear. These influencing parameters may include, for example, length of various anatomical features, the weight of the user, proprioceptive control by the user, strength of the user, and flexibility of the user. Traditionally, manufacturers of these devices tend to only focus on body weight and subjective assessment of activity level of the users with height of the user sometimes playing a role in the selection of the device.

The present disclosure allows for an assessment of the human-device interaction and keeps all parameters in mind when selecting the device, training on the device, or customizing the device to a particular user. As parameters change, such as in the relative strength of the user, weight of the user, height of the user, changes in stability and proprioception, and others as an individual progresses through, for example, rehabilitation, it is important to assess and re-assess on a regular basis to ensure that the appropriate device is still the one being used by the individual.

In one aspect, methods of using an inertial measurement unit (IMU) sensor for collection of biomechanical data are disclosed. In one embodiment, the method includes: placing the IMU sensor onto an anatomical portion of a user; collecting data from the IMU sensor during a prescribed exercise being performed by the user; processing the data from the IMU sensor using a machine learning or artificial intelligence engine to determine a type of an assistive device for use with the user; collecting additional data from the IMU sensor when the user is using the assistive device; processing the additional data using the machine learning or artificial intelligence engine to determine an exercise to be performed by the user when using the assistive device; and displaying the additional data on a graphical user interface (GUI) along with previously collected data for another performance of the exercise.

In one variant, the processing of the additional data using the machine learning or artificial intelligence engine further includes determining adjustments to the assistive device to further optimize assistive device performance.

In another variant, the determining of the adjustments to the assistive device includes determining dimension adjustments for the assistive device.

In yet another variant, the determining of the adjustments to the assistive device includes determining a strap configuration for the assistive device.

In yet another variant, the determining of the adjustments to the assistive device includes determining a wedge type for use with the assistive device.

In yet another variant, the determining of the adjustments to the assistive device includes determining material types for the assistive device.

In yet another variant, the method further includes iteratively collecting additional data from the IMU sensor during additional performances of the exercise performed by the user when using the assistive device and determining a different exercise to be performed by the user using the machine learning or artificial intelligence engine.

In yet another variant, the method further includes placing an additional IMU sensor onto the assistive device.

In yet another variant, the iteratively collecting of the additional data includes iteratively collecting data from both the IMU sensor and the additional IMU sensor.

In yet another variant, the data collected from both IMU sensor and the additional IMU sensor includes acceleration data and angular velocity data stored in a quaternion number system and the method further includes converting the acceleration data and the angular velocity data from the quaternion number system into three-dimensional (3D) Euler angle data prior to the displaying of the additional data on the GUI.

In yet another variant, the data collected from the IMU sensor includes acceleration data and angular velocity data stored in a quaternion number system and the method further includes converting the acceleration data and the angular velocity data from the quaternion number system into three-dimensional (3D) Euler angle data prior to the displaying of the additional data on the GUI.

In yet another variant, the method further includes sub-dividing the acceleration data and the angular velocity data as a function of gait cycle for the user, where the gait cycle is defined as a heel strike by the user followed by an immediately succeeding heel strike by the user.

In yet another variant, the sub-dividing of the acceleration data and the angular velocity data is performed prior to the displaying of the additional data on the GUI.

In yet another variant, the displaying of the previously collected data for another performance of the exercise further includes displaying data of use of different types of assistive devices during prior performance of the exercise.

In yet another variant, the displaying of the previously collected data for another performance of the exercise includes displaying data of use of a different configuration for the assistive device.

In yet another variant, the displaying of the previously collected data for another performance of the exercise includes displaying data from other users using the type of the assistive device.

In yet another variant, the displaying of the previously collected data for another performance of the exercise includes displaying a shaded area, the shaded area being control values collected from other users.

In yet another variant, the displaying of the previously collected data for another performance of the exercise includes displaying personal best data for the user of the assistive device.

In yet another variant, the method further includes determining a different type of exercise to be performed by the user of the assistive device using the machine learning or artificial intelligence engine.

In yet another variant, the method further includes collecting additional data from the IMU sensor when the user is using the assistive device and performing the different type of exercise and displaying the additional data on the GUI along with previously collected data for another performance of the different type of exercise.

In another aspect, methods to assess, report on and train the effectiveness and efficiency of use of the assistive device and/or supporting gear by the user are disclosed.

In yet another aspect, computer readable apparatus for executing the aforementioned methodologies are disclosed.

In yet another aspect, systems for implementing the aforementioned methodologies are disclosed.

Other features and advantages of the present disclosure will immediately be recognized by persons of ordinary skill in the art with reference to the attached drawings and detailed description of exemplary implementations as given below.

### BRIEF DESCRIPTION OF DRAWINGS

The features, objectives, and advantages of the present disclosure will become more apparent from the detailed description set forth below when taken in conjunction with the drawings, wherein:

FIG. 1 is an exemplary system for use with the IMU (inertial measurement unit) sensors of FIG. 2A, in accordance with the principles of the present disclosure.

FIG. 2A are exemplary IMU sensors, in accordance with the principles of the present disclosure.

FIG. 2B are exemplary IMU sensor placement options for both the anterior and posterior portions of the human body, in accordance with the principles of the present disclosure.

FIG. 2C is an exemplary IMU sensor placement on an ankle foot orthosis (AFO), in accordance with the principles of the present disclosure.

FIG. 3A is a logical block diagram of an exemplary reinforcement learning agent, in accordance with the principles of the present disclosure.

FIG. 3B is a logical flow diagram for using the exemplary system of FIG. 1, in accordance with the principles of the present disclosure.

FIG. 4A is exemplary IMU sensor data broken down by gait cycle, in accordance with the principles of the present disclosure.

FIG. 4B is exemplary IMU sensor data broken down by gait cycle in order to determine push-off, in accordance with the principles of the present disclosure.

FIG. 5 is a logical diagram of information flow between in-clinic and home training using the exemplary system of FIG. 1, in accordance with the principles of the present disclosure.

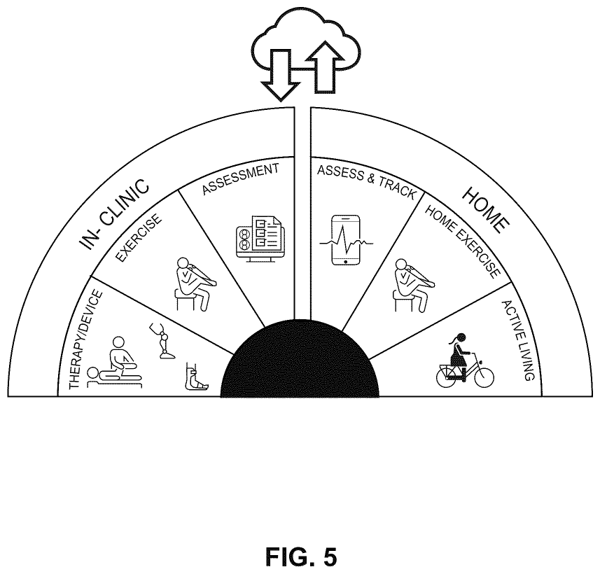

FIG. 6A is an exemplary graphical user interface illustrating the display of data collected from the one or more IMU sensors of FIG. 2A to assess user performance and utilization in a sit-to-stand activity, in accordance with the principles of the present disclosure.

FIG. 6B is an exemplary graphical user interface illustrating the data collected from the one or more IMU sensors of FIG. 2A to assess user performance and utilization in a walking activity, in accordance with the principles of the present disclosure.

FIG. 7 is a logical diagram of continuous utilization of the exemplary system of FIG. 1, in accordance with the principles of the present disclosure.

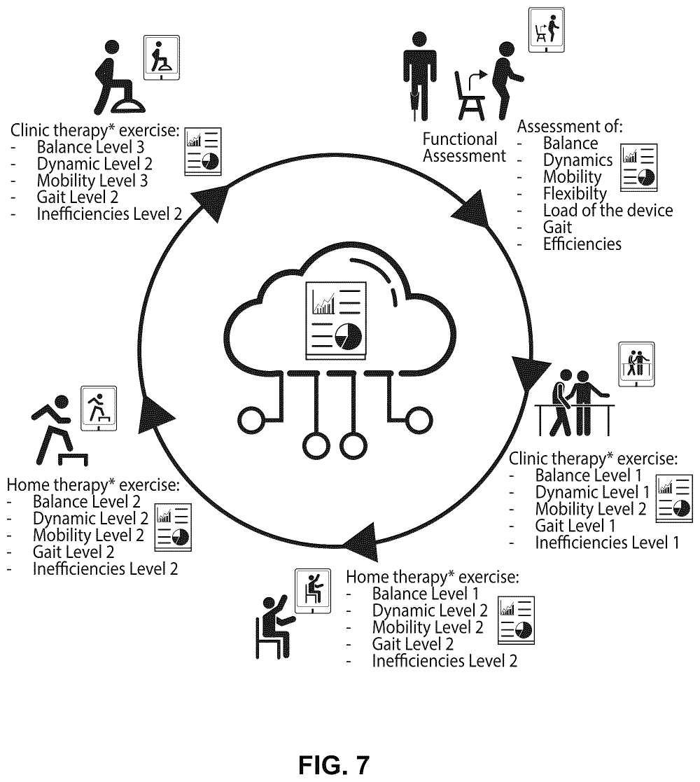

FIG. 8 is an exemplary system for use in a community level approach for using the exemplary system of FIG. 1, in accordance with the principles of the present disclosure.

All Figures disclosed herein are @ Copyright 2023-2024 KIM EHF. All rights reserved.

### DETAILED DESCRIPTION

### Overview

Assistive devices (e.g., prosthetic, and orthotic devices used for people with underlying medical conditions and/or disabilities) and/or supporting gear (e.g., devices that are used for the training of able-bodied athletes) are often intended to assist the human body achieve certain levels of capability of performance when a user has, for example, an underlying weakened or abnormal joint(s) or limb(s). For example, supporting research indicates that exercise, training, and rehabilitation can have positive impacts on allowing the user of the assistive device and/or supporting gear to achieve certain levels of capability of performance. However, to achieve optimized use of these devices, it is important to assess various parameters that help determine the qualitative performance of the user wearing these devices at any given time, as well as provide feedback to the user during use of these devices to assess various objective parameters, indicators, and performance indexes. To this end, prescribed sets of exercises and tailored training activities can positively influence the performance of the user with the use of these devices, especially when these exercises and activities are correctly executed.

Methodologies described herein can be used to assess the overall user capability and/or quality of performance of these exercises and activities through visual, haptic and/or other feedback methodologies. For example, once these exercises and activities have been performed and compliance has been tracked, the user will be prompted through a series of performance related indexes. As a result, a user's performance using these devices can either: (1) be improved through challenge-point training; or (2) might indicate a need for utilization of more advanced types of assistive devices or supporting gear; or (3) might objectify regression and need for intensified therapy or training. Solutions provided by the present disclosure solve several related problems. First, the present disclosure assists with the assessment of the efficiency and effectiveness of use of the assistive device and/or supportive gear. Second, the present disclosure assists with the optimization of the efficiency and effectiveness of use of the assistive device and/or supportive gear. Third, the present disclosure provides guidance as to the selection of the type of assistive device and/or supportive gear to be utilized. The solutions provided herein are a novel combination of hardware (e.g., computing devices, visual displays, haptic feedback devices, and motion sensors) and software where efficiency and effectiveness parameters are monitored and tracked.

### Exemplary Embodiments

Detailed descriptions of the various embodiments and variants of the apparatus and methods of the present disclosure are now provided. It is noted that wherever practicable similar or like reference numbers may be used in the figures and may indicate similar or like functionality. The figures depict embodiments of the disclosed system (or methods) for purposes of illustration only. One skilled in the art will readily recognize from the following description that alternative embodiments of the structures and methods illustrated herein may be employed without necessarily departing from the principles described herein.

While primarily discussed in the context of the application of the present disclosure to assistance of correction of gait abnormalities, or optimizing the gait of an athlete, it is not necessarily a prerequisite for the present technology to be utilized solely for lower body weakened joint(s) and/or limb(s). For example, it would be readily appreciated by one of ordinary skill given the contents of the present disclosure that the techniques and methodologies described herein may be applied to, for example, upper body performance, resulting from, for example, weakened joint(s) and/or limb(s). These and other variants would be readily apparent to one of ordinary skill given the contents of the present disclosure.

### Exemplary Assessment and Progress Tracking Systems—

The present disclosure relates to the field of assessment and enhancement of human performance for wearers of assistive devices (e.g., prosthetics) and/or supporting gear by providing methods, systems and software for assessing various parameters in multiple dimensions in order to quantify the efficiencies of a user's wearing of these aforementioned assistive devices and/or supporting gear. In particular, the system 100 objectifies the performance of the user of the aforementioned assistive devices and/or supporting gear in terms of parameters such as, for example, stability, movement control, balance, and energy efficiency. Exercises, which are provided to the user, are monitored by the system 100 and outcomes are interpreted using a challenge point management approach to allow for movement and/or performance optimization for the user. The system 100 may also utilize so-called reference data which may include standardized performance data such as, for example, average performance, optimal performance, and personal best performance for these exercises. The system 100 may also use the data that it monitors to assist with the selection of the appropriate assistive devices and/or supporting gear based on the assessment of the capabilities and other physical or biomechanical characteristics of the user.

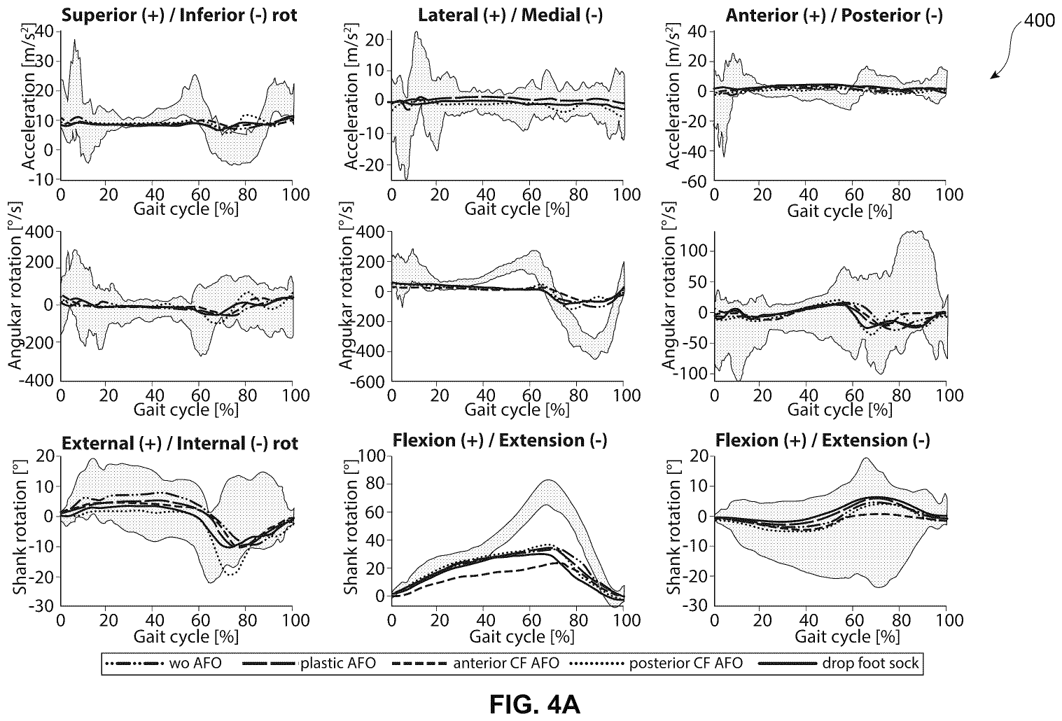

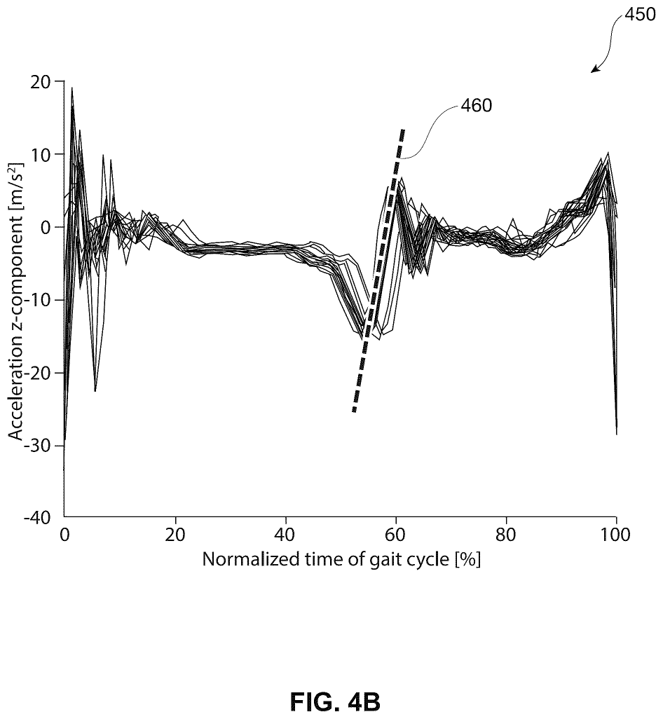

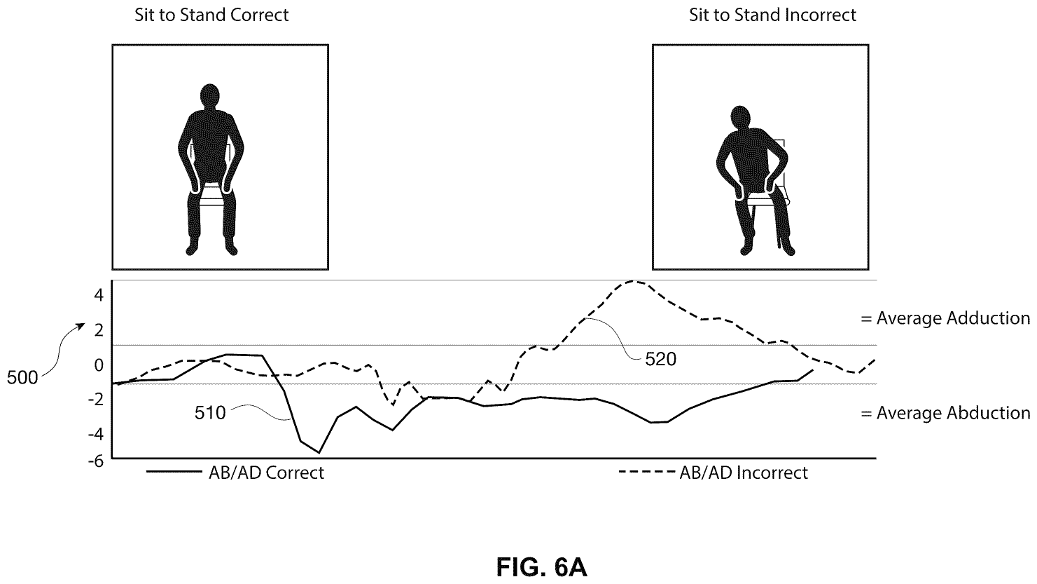

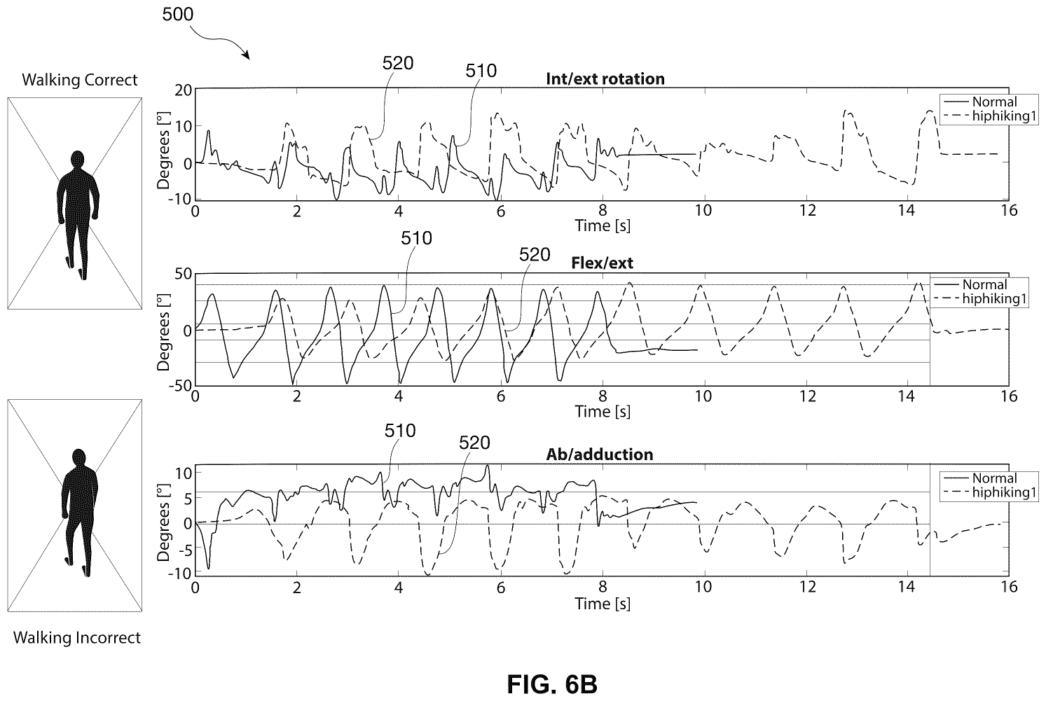

Graphical and/or numerical representation of this performance (see also FIGS. 4A-4B and 6A-6B) including, for example, stability, movement control, balance, and energy efficiency is provided to the user as feedback on the user's performance (whether provided to the user directly and/or to treating clinicians) when using these assistive devices and/or supporting gear. For example, this feedback can be used to justify modifications to these assistive devices and/or supporting gear, provide recommendations on upgrades or replacement of these assistive devices and/or supporting gear if deemed necessary due to, for example, user progression, user regression, or user stagnation. In some implementations, haptic and/or tactile feedback can be provided which assists the user when using these assistive devices and/or supporting gear in daily life or when performing exercises from the training program. In other words, the system 100 provides a reliable method to track user compliance with regards to use of the assistive devices and/or supporting gear and recommended exercises or training protocols. Performance tracking and assessment can be done against, for example, baseline performance, individual personal best performance, ‘normal’ performance or expert performance by others. Tracking can also be done in an online community where users of the same or similar devices are able to share and compare data at will and compete in compliance efforts for these exercise programs.

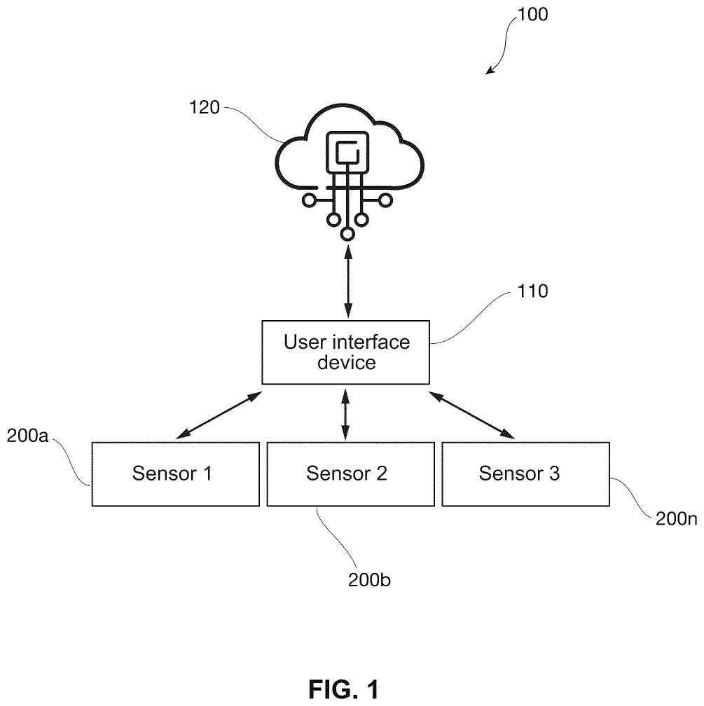

Referring now to FIG. 1, one exemplary system 100 for assessment and progress tracking of a user who utilizes assistive devices and/or supporting gear is shown and described in detail. The system 100 may include one or more sensors 200a, 200b, . . . 200n, one or more user interface device(s) 110, as well as one or more server(s) 120. As a brief aside, the functionality of the various modules described herein may be implemented through the use of software executed by one or more processors (or controllers) and/or may be executed via the use of one or more dedicated hardware modules. The computer code (software) disclosed herein is intended to be executed by a computing system (e.g., the one or more server(s) 120 and/or the user interface device 110) that is able to read instructions from a non-transitory computer-readable medium and execute them in one or more processors (or controllers), whether off-the-shelf or custom manufactured. The computing system may be used to execute instructions (e.g., program code or software) for causing the computing system to execute the computer code described herein. In some implementations, the computing system operates as a standalone device or a connected (e.g., networked) device that connects to other computer systems. The computing system may include, for example, a personal computer (PC), a tablet PC, a notebook computer, a smart phone, or other custom device capable of executing instructions (sequential or otherwise) that specify actions to be taken. In some implementations, the computing system may include a server 120. In a networked deployment, the computing system may operate in the capacity of a server 120 or client (e.g., user interface device 110) in a server-client network environment, or as a peer device in a peer-to-peer (or distributed) network environment. Moreover, a plurality of computing systems may operate to jointly execute instructions to perform any one or more (or subsets) of the methodologies discussed herein.

An exemplary computing system includes one or more processing units (generally processor apparatus). The processor apparatus may include, for example, a central processing unit (CPU), a graphics processing unit (GPU), a digital signal processor (DSP), a controller, a state machine, one or more application specific integrated circuits (ASICs), one or more radio-frequency integrated circuits (RFICs), or any combination of the foregoing. The computing system also includes a main memory. The computing system may include a storage unit. The processor, memory and the storage unit may communicate via a bus.

In addition, the computing system may include a static memory, and a display driver (e.g., to drive a plasma display panel (PDP), a liquid crystal display (LCD), a projector, or other types of displays). The computing system may also include input/output devices such as an alphanumeric input device (e.g., touch screen-based keypad or an external input device such as a keyboard), a dimensional (e.g., 2-D or 3-D) control device (e.g., a touch screen or external input device such as a mouse, a trackball, a joystick, a motion sensor, or other pointing instrument), a signal capture/generation device (e.g., a speaker, camera, and/or microphone), and a network interface device, which may also be configured to communicate via the bus.

Embodiments of the computing system corresponding to a client device (e.g., a user interface device 110) may include a different configuration than an embodiment of the computing system corresponding to a server 120. For example, an embodiment corresponding to a server 120 may include a larger storage unit, more memory, and a faster processor but may lack the display driver, input device, and dimensional control device. An embodiment corresponding to a client device 110 (e.g., a personal computer (PC) or smartphone) may include a smaller storage unit, less memory, and a more power efficient (and slower) processor than its server 120 counterpart(s).

The storage unit includes a non-transitory computer-readable medium on which is stored instructions (e.g., software) embodying any one or more of the methodologies or functions described herein. The instructions may also reside, completely or at least partially, within the main memory or within the processor (e.g., within a processor's cache memory) during execution thereof by the computing system, the main memory and the processor also constituting non-transitory computer-readable media. The instructions may be transmitted or received over a network via the network interface device.

While non-transitory computer-readable medium is shown in an example embodiment to be a single medium, the term “non-transitory computer-readable medium” should be taken to include a single medium or multiple media (e.g., a centralized or distributed database, or associated caches and servers) able to store the instructions. The term “non-transitory computer-readable medium” shall also be taken to include any medium that is capable of storing instructions for execution by the computing system and that cause the computing system to perform, for example, one or more of the methodologies disclosed herein.

Portions of the system 100 of FIG. 1 may be located proximate to one another, while other portions may be located remote from some of the portions. For example, the sensors 200 and user interface device 110 may be located on the premises for the user of the assistive device and/or supporting gear, while the server 120 may be located remote from the user (e.g., within the “cloud”). In some implementations, the sensor(s) 200, user interface device 110, and server 120 may be located at the premises of the user of the assistive device and/or supporting gear. These and other variants would be readily apparent to one of ordinary skill given the contents of the present disclosure.

In some implementations, the user will install an application on an exemplary user interface device 110 located within, for example, the user's residence. This exemplary user interface device 110 may access a remote computing system 120 (e.g., a computing system resident in the cloud) that implements some or all of the exemplary functionality disclosed herein. For example, the user may capture user assessment and progress tracking data on a local device. This data may then be transmitted over a network (e.g., the Internet) to the remote computing system 120 (e.g., resident within the cloud), where the progress tracking data is processed. This processed tracking data may then be transmitted back to a local computing system for display. These and other variants would be readily apparent to one of ordinary skill given the contents of the present disclosure.

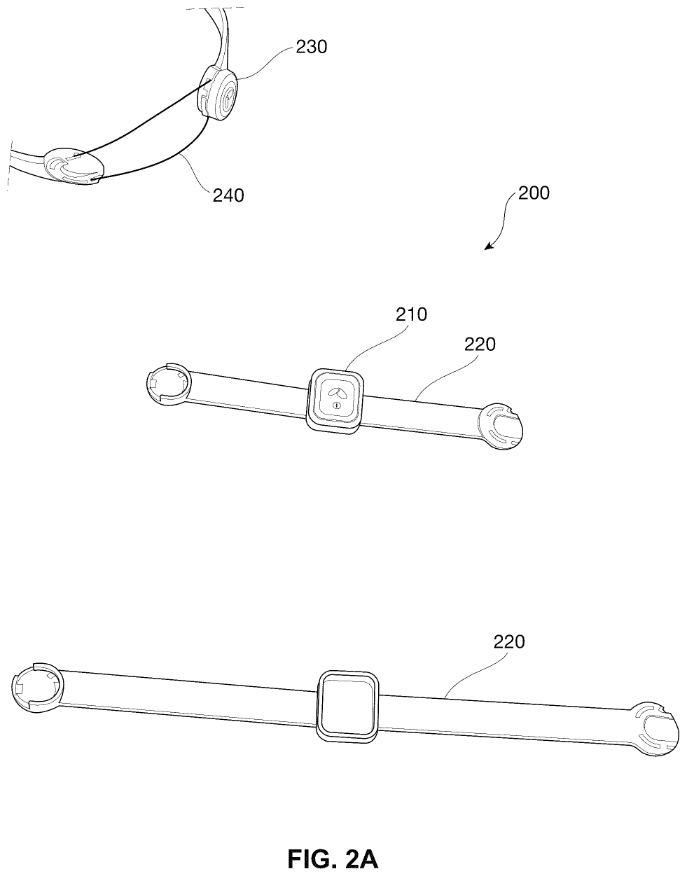

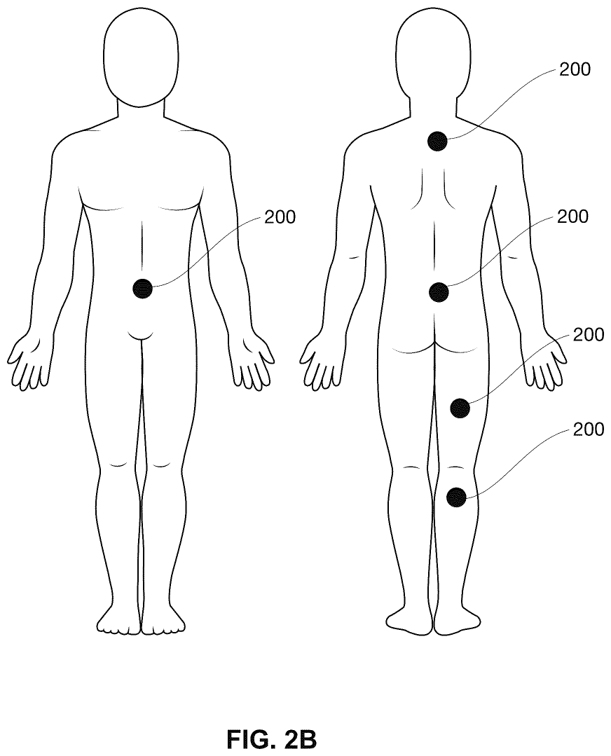

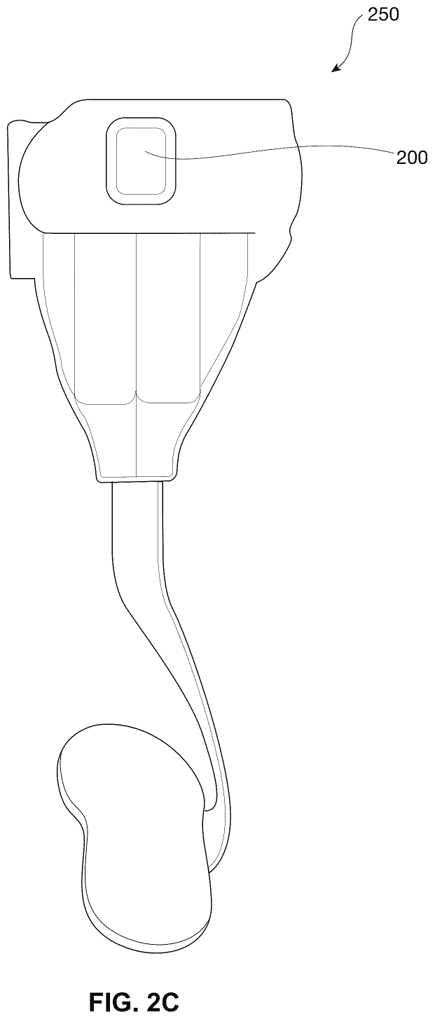

Referring now to FIG. 2A, an exemplary sensor 200 for use with the system 100 is shown and described in detail. The sensor 200 may include a single-dimensional or three-dimensional accelerometer 210, a fixed length strap 220 as well as a strap adjustment mechanism 230, 240. As a brief aside, accelerometers are typically electromechanical transducers that produce an electrical output that is proportional to the acceleration in which the accelerometer is being subjected. Moreover, three-dimensional accelerometers typically consist of three (3) individual accelerometers that are mounted in a single housing, with each of the three (3) individual accelerometers being mounted, for example, orthogonal with respect to one another. Accordingly, these three-dimensional accelerometers can not only detect acceleration, but also detect individual acceleration in the three planes in which each of these three (3) accelerometers are oriented. The data generated and stored by these sensors 200 may include acceleration data (e.g., in m/s²) as well as angular velocity data (e.g., in°/s). In some implementations, this acceleration data and angular velocity data is stored in a quaternion number system. These three-dimensional accelerometers 210 may also include a wireless network interface which outputs the three-dimensional acceleration measurements being recorded (e.g., quaternion data) by the three-dimensional accelerometer 210 to, for example, the user interface device 110, though wired network interfaces may be utilized in some implementations.

The fixed length strap 220 may be sized in accordance with the anatomical area upon which the sensor 200 is to be mounted. For example, the upper sensor 200 illustrated in FIG. 2A may be adapted for smaller anatomical features of the human anatomy, while the lower sensor 200 illustrated in FIG. 2A may be adapted for larger anatomical features (as compared with the upper sensor) of the human anatomy. The sensors 200 may also include a strap adjustment mechanism 230, 240 to accommodate a larger differential in terms of anatomical feature dimension differences from person to person. As shown in FIG. 2A, this adjustment mechanism includes a rotary tensioning mechanism 230 as well as a cable 240. Accordingly, by tightening (or loosening) the rotary tensioning mechanism 230, the sensor 200 can be adapted to fit a particular user. While a specific adjustment mechanism 230, 240 is shown, it would be readily apparent to one of ordinary skill that the specific adjustment mechanism shown may differ in some implementations. For example, known adjustment mechanisms such as those found on, for example, a belt (e.g., a belt buckle) or other types of adjustment mechanisms may be readily substituted in alternative implementations. Moreover, in some implementations, the strap 220 length may be individually customizable to fit a particular user's anatomy. Additionally, in some implementations it may be desirable to mount the three-dimensional accelerometer 210 directly to the user's assistive device and/or supporting gear as well as the user's clothing. For example, the three-dimensional accelerometer 210 may reside in a pocket contained in the user's assistive device and/or supporting gear, or in their clothing. These and other implementations would be readily apparent to one of ordinary skill given the contents of the present disclosure.

Referring now to FIG. 2B, exemplary positioning of the sensors 200 with relation to the human anatomy is shown. For example, one sensor 200 may be placed over the user's belly button, one sensor 200 may be placed on the user's upper back along the centerline of the user, one sensor 200 may be placed on the user's lower back along the centerline of the user, while another sensor 200 may be placed on the thigh of the user, and another sensor 200 may be placed on the lower leg of the user. While a specific implementation is shown in FIG. 2B, it would be readily apparent to one of ordinary skill given the contents of the present disclosure that more (or less) sensors 200 may be utilized in some implementations. Additionally, while specific locations have been described with reference to FIG. 2B, it would be readily apparent to one of ordinary skill given the contents of the present disclosure that alternative placement of the sensor 200 (on e.g., the upper and/or lower arm) may be utilized dependent upon the parameters that are desired to be captured by the sensor 200. FIG. 2C exemplifies the integration of a sensor 200 directly into an assistive device such as an AFO 250.

These sensors 200 are configured to capture any number of different biomechanical parameters. For example, these sensors 200 may capture: (1) peak acceleration at toe-off as a representation of energy at push-off; (2) vertical displacement versus toe lever as a representation of foot flexibility versus stiffness; (3) midstance stop of posterior movement as an indication of toe stiffness; (4) peak acceleration at impact as a representation of walking speed; (5) energy ratio (peak acceleration at impact as a function of peak acceleration at toe-off) to define the efficiency of the device-corrected by the vertical displacement; (6) Medio Lateral (ML) displacement as an indication for stability; (7) Antero Posterior (AP) trajectory as a representation of the AP roll-over smoothness; (8) tibial tilt as a representation of alignment neutrality; and (9) rotational movement in stance and swing as a representation of stability. See also FIGS. 4A and 4B discussed infra.

By capturing this data from the sensors 200 and transmitting this captured data to, for example, a user interface device 110, the system 100 may identify suboptimal performance by the user when wearing the assistive device and/or supporting gear. Consequently, challenge point managed training programs may then be provided to the user via, for example, the user interface device 110 to increase the effectiveness and efficiency of the use of these assistive devices and/or supporting gear. The user interface device 110 may also store this captured data in memory, thereby allowing, for example, the treating clinician (or the user) to compare this captured data to, for example: (1) previously captured data for the user; (2) reference data from other users; (3) personal best data for the user (and/or other users); or (4) performance of the user on a previous or alternative device. Accordingly, this information may be used for further adjustment of the training programs offered (e.g., different exercises) or even different assistive device and/or supporting gear selection. In other words, the system 100 solves several related problems. First, the system 100 assesses the efficiency and effectiveness of use of the assistive device and/or supporting gear. Second, the system 100 optimizes the efficiency and effectiveness of use of the assistive device and/or supporting gear. Third, the system 100 advises on the selection of the most beneficial assistive device and/or supporting gear. In other words, the system 100 provides qualitative insight into the use of the assistive device and/or supporting gear rather than quantitative data that has been limited to frequency (or duration) of use. In some implementations, the user interface device 110 includes software which receives the quaternion data from the sensors 200 and converts this quaternion data to three-dimensional (“3D”) Euler angle data. As a brief aside, conversion of quaternion data to 3D Euler angle data may be beneficial as the 3D Euler angle data may be more intuitive to, for example, treating clinicians. This primarily is a result of the fact that quaternion number system data may be more efficient from a data storage and processing perspective; however, the quaternion number system data is a four-dimensional (“4D”) representation of 3D movement.

In some implementations, the user interface device 110 displays basic activities for the user to perform while wearing the assistive device and/or supporting gear. A sensor 200, which may be placed on the assistive device and/or supporting gear (or on other portions of the user's anatomy such as that shown in FIG. 2B) transmits real-time data from the sensor 200 to the user interface device 110 and displays this data in a manner that provides audio, visual, tactile and/or graphical feedback on these measured parameters. For example, a normal range of motion may be displayed on the display of the user interface device 110 and feedback is provided as to how the user is performing in relation to this normal range of motion. In some implementations, this normal range of motion may vary from optimal range as described for able-bodied individuals, to personal best performance of the individual using the assistive device and/or supporting gear or average range for users of comparable assistive devices.

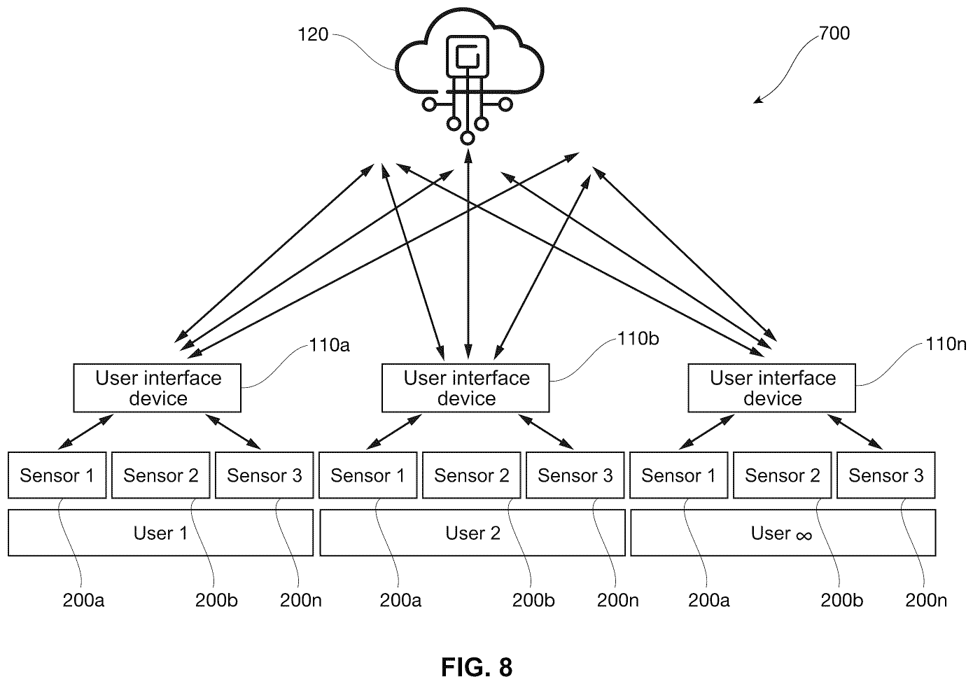

FIG. 8 illustrates such a system 700 where multiple users of comparable assistive devices share their data with one or more servers 120. Such an architecture may provide for an interactive community which is collecting and sharing data amongst the users in a particular group. This architecture may be beneficial as an interactive community can foster competitiveness for users of these assistive devices and/or supporting gear trying to outperform parameters while optimizing their outcomes.

Parameters for making training progressively more difficult may lie within a series of physical exercises projected on a display (e.g., on a display on the user interface device 110) while the user is real time connected to the system 100. Displayed exercises may come in different degrees of difficulty and the parameters tracked may be projected on a graphical user interface. As exercises get more challenging, target ranges for the tracked parameters may start larger, and may progressively become narrower as the user improves. Comparison may be made to expert users or research-defined normal ranges. In some of the exercises, focus may be on only one of the parameters, however; more challenging exercises may track multiple parameters during the exercise. New exercises and new parameters may be added. The system 100 may also make recommendations on the level of difficulty of the exercises based on previous assessments or progress in execution of the exercise programs. The sensors 200 may generate three-dimensional parameters for the assessment and provision of feedback as described elsewhere herein. In other implementations, the sensors 200 may generate two-dimensional parameters to assess and provide feedback in some implementations.

Various exercises may be displayed to the user via the user interface device 110. For example, the exercises that are displayed may include, without limitation: (1) a stable seated position with weight shifting in the medio-lateral direction; (2) unstable seated position with weight shifting in the medio-lateral direction; (3) double leg stance with weight shifting in the medio-lateral direction; (4) double leg stance with weight shifting in the anterior-posterior direction; (5) single leg stance with shifts in leg inclination in the medio-lateral direction; (6) single leg stance with shifts in leg inclination in the anterior-posterior direction; (7) any of the aforementioned positions where the contralateral leg is lifted off the ground and performs an exercise; (8) single leg stance with rotational movement of the stance leg; (9) side walking exercises with front steps; (10) side walking exercises with posterior steps; (11) stability exercises on a stable surface; and (12) stability exercises on an unstable surface. The user may also use the system 100 in a private mode where the results are only available to the user, or the user may use the system 100 in a public or semi-public mode where the user can connect to other users around the world and add a competitive aspect to the rehabilitation.

During these exercises the user may not always have to look at a screen (e.g., the screen of the user interface device 110) for visual feedback. For example, the user may receive haptic feedback during execution of activities or subsets of such activities. As but one non-limiting example, when a user walks with an assistive device a certain level of anterior lean of the lower leg towards terminal stance followed by a certain level of acceleration of the lower leg after push-off is expected. In such a scenario, the sensor(s) 200 being worn by the user may provide a vibratory response to the user depending on how the user is performing the action of the exercise or activity. For example, if the user does not lean sufficiently forward while taking a step, a predetermined type of haptic feedback is provided to let the patient know that the optimal level of anterior movement was not attained. Additionally, a different type of haptic feedback (e.g., a different vibratory response) may be provided to let the user know that a sufficient movement has been achieved. Various gradations in a user's ability to complete a certain task may be provided using three (3) or more different type of haptic responses.

The system 100 may also utilize recognized clinical assessment tests and objective tests for those using these assistive devices and/or supporting gear such as: (1) Amputee Mobility Predictor (pro/no pro); (2) time up and go test; (3) two minute and/or six minute walking test; and (4) L-test. Questionnaires and/or self-reporting tools for those using these assistive devices and/or supporting gear may be provided to, for example, the user interface device 110 and may include: (1) ABC UK: Activities-specific Balance confidence scale-UK, which is a self-report, quality of life outcome measure, relating balance confidence to functional activities; (2) PEQ: The Prosthesis Evaluation Questionnaire is used to describe the perception of difficulty in performing prosthetic function and mobility. It is a self-report, 82-item questionnaire developed to assess prosthetic function, mobility, psychosocial aspects, and well-being; (3) LCI: Locomotor capability index questionnaire: the LCI is a self-report outcome measure that forms part of the Prosthetic Profile of the Amputee questionnaire. The LCI assesses a lower limb amputee's perceived capability to perform fourteen (14) different locomotor activities with a prosthesis; (4) LCI: The Locomotor Capability Index questionnaire is a self-report outcome measure that forms part of the Prosthetic Profile of the Amputee questionnaire. The LCI assesses a lower limb amputee's perceived capability to perform 14 different locomotor activities with a prosthesis; (5) TAPES: The Trinity Amputation and Prosthesis Experience Scale is used to examine psychosocial issues related to adjustment to a prosthetic, specific demands of wearing a prosthesis and potential sources of maladjustment; (6) The Barthel scale or Barthel ADL index is an ordinal scale used to measure performance in activities of daily living (ADL). Each performance item is rated on this scale with a given number of points assigned to each level or ranking; and (7) The Prosthetic Profile of the Amputee (PPA) measures the function of adult unilateral lower limb amputees (prosthetic users and nonusers) in terms of predisposing, enabling, and facilitating factors related to prosthetic use after discharge from the hospital.

### Exemplary Artificial Intelligence Engine—

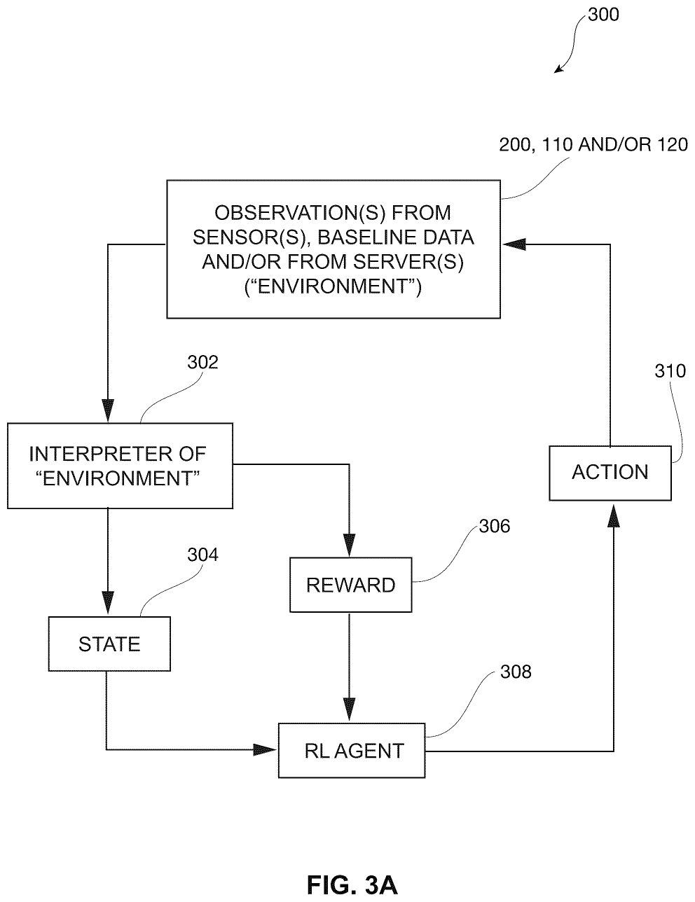

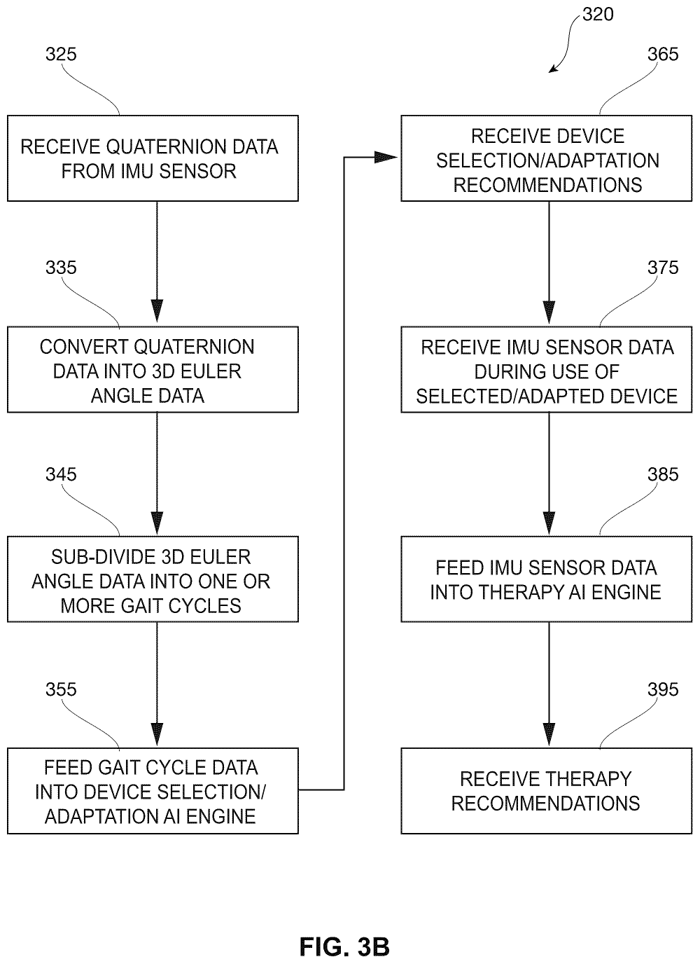

Referring now to FIG. 3A, a reinforcement learning (RL) system 300 may be utilized to implement the various methodologies and systems 100 described herein. As will be described in additional detail with regards to FIG. 3A, a RL agent 308 may take so-called action(s) 310 in response to observations being made (e.g., by the sensors 200, the user interface device 110; and/or servers 120). For example, the RL agent 308 may receive assessment feedback from the sensors 200 in response to a first training regimen provided to a user and in response, may provide to the user a second training regimen. This second training regimen may be objectively more difficult to perform than the first training regimen; however, this second training regimen may be demonstrated to improve a user's ability to use a particular assistive device and/or supporting gear. As but another example, the RL agent 308 may suggest to a user a second training regimen that is less difficult to perform in some implementations, in response to observations made of the first training regimen. For example, a given training regimen may result in suboptimal outcomes for the user in a given area and a second training regimen may be displayed which has been demonstrated to improve on that given suboptimal outcome.

As a brief aside, RL is an area of machine learning that utilizes software agents that take actions in an environment in order to maximize the notion of cumulative reward. In other words, RL can not only maximize immediate rewards resultant from immediate actions, RL can also maximize long term rewards while taking a series of actions with less immediate reward impact through the application of the concept known as discounted rewards. The RL agent's environment (e.g., data from the sensors 200, user interface device 110 and/or server(s) 120) is the set of all observations made in response to, for example, a given training regimen. The goal of the RL agent 308 is to take the best action 310 (e.g., to provide the most correct set of training regimens for improvement of use of the selected assistive device and/or supporting gear) based on the environment that the RL agent 308 observes.

The RL agent 308 utilizes an interpreter of the environment 302 that provides a so-called state 304 to the RL agent 308. The state 304 in this context is defined by the set of observations that make up the environment. The interpreter 302 may consist of software (i.e., a computer program and/or dedicated hardware) that assembles the environment into a format that the RL agent 308 can utilize in order to take actions 310. The RL agent's 308 action 310 selection is modeled as a map called a policy. The defined policy may be thought of as a map between the observations made and the actions taken based on these observed observations. The policy map may give the probability of taking a given action when in a given state in some implementations.

The RL agent 308 is also defined by a state-value function that defines the expected return (or reward 306) when successively following a defined policy. The initial policy and state value function can be defined based on historical training outcomes from qualified trained personnel (e.g., clinicians and/or trainers). However, this initial policy and state value function can be updated over time as the RL agent 308 takes further actions 310 and collects additional rewards 306 (e.g., via the reward value function for the RL agent 308) for these actions 310. These rewards 306 may be characterized by soliciting feedback from the system 100 as a user progresses through a given training regimen.

Additionally, or alternatively, from the training regimen recommendation, the RL agent 308 may also assist with determining the types of assistive devices and/or supporting gear to be recommended to a given user as well as provide recommendations regarding the fine-tuning of the assistive device and/or supporting gear for optimized usability and performance. For example, the RL agent 308 may make recommendations on the selection of the optimal assistive device for a user, based on, for example, their ability to walk without a device, or their ability to walk based on a basic device. The RL agent 308 may also include recommendations on dimensions for a given device, materials for the device, and/or other biomechanical characteristics of the device such as, for example, various dimensions, stiffness, levels of support, etc., which would be supported by the objectively measured data while the user is using the device. After the RL agent 308 has made its recommendation regarding the specific type of assistive device and/or supporting gear, the RL agent 308 may then make data-driven recommendations on the set-up and/or fine tuning of the device by means of, for example, wedges, straps, adjustable calf-pieces and other adaptable features which will further optimize the performance of the user with the device. Over time, through repeated assessments, the RL agent 308 may further make recommendations on the need for device upgrades, downgrades, or changes to the type of device.

The RL agent 308 may also make recommendations on the optimal focus of the therapeutic exercises to further enhance the performance of the user on the recommended assistive device and/or supporting gear. Exercise focus can include, for example, balance exercises, gait training, strength and stability exercises, and/or proprioceptive training. The RL agent 308 may also derive the optimal mix of the most appropriate exercises, as well as the optimal frequency of exercises to best support the rehabilitation process of the user. In some implementations, a therapist fulfills the role of supervisor for correct execution of the recommended exercises and may implement regular assessments into the therapy program of the user of the system. These regular assessments create a continuous data-collection flow and a feedback loop feeding into the further optimization of the system over time. While the use of RL as a means of progressing a user through selection and/or use of an exemplary assistive device and/or supporting gear, it would be readily apparent to one of ordinary skill given the contents of the present disclosure that alternative artificial intelligence and/or machine learning paradigms may be substituted in favor of the aforementioned RL, in some implementations.

Referring now to FIG. 3B, an exemplary logical flow diagram 320 for using the aforementioned system 100 (and the aforementioned RL system 300 or alternative artificial intelligence and/or machine learning paradigms) is shown and described in detail. At step 325, quaternion data is received from one or more inertial measurement unit (“IMU”) sensors 200. This quaternion data may be captured at a rate of, for example, 60 Hz and may include acceleration data (e.g., in m/s²) and angular velocity data (e.g., in°/s). As described supra, the quaternion data captured from these IMU sensors 200 may be more efficient from a data storage and processing perspective.

At step 335, the received quaternion data is converted into 3D Euler angle data which may be more intuitive for users as well as the treating clinicians that are supporting these users. At step 345, the 3D Euler angle data is sub-divided into one or more gait cycles. As a brief aside, the gait cycle as described herein may be defined at a first heel strike as 0%, followed by a subsequent heel strike as 100%. The heel strike may be advantageous as it is easily determinable through analysis of the data. For example, the heel strike may be determined by finding the zero point of the y-component (y-direction pointing outwards from the body) of the angular velocity in conjunction with the highest flexion angle. In other words, immediately prior to the heel encountering the ground, the leg swings forward creating a relatively large rotational velocity, as compared to other portions of the gait cycle and especially in relatively healthy subjects. Additionally, when the heel strikes the ground, a spike in acceleration (or deceleration) due to the force required to bring the foot from the swing to stopping on the ground. In short, in order to determine when the heel hits the floor, the angular velocity becomes zero after the swing phase, the rotational angle of the shank being around the maximum value, and a spike in the acceleration (or deceleration) signal.

Referring now to FIG. 4A, various plots using the data received from the IMU sensors 200 may be derived. For example, acceleration as a function of gait cycle may be determined in the superior/inferior directions, the lateral/medial directions, and the anterior/posterior directions. Additionally, angular rotation as a function of gait cycle may be determined in the superior/inferior directions, the lateral/medial directions, and the anterior/posterior directions, as well as external/internal rotations, flexion/extension, as well as adduction/abduction direction. Each of these data sets may include, for example, data associated with a given user not using an ankle foot orthosis (“AFO”), a given user using a plastic AFO, a given user using an anterior carbon fiber (“CF”) AFO, a given user using a posterior CF AFO, and a given user using a drop foot sock, etc. These use cases are merely exemplary, and other forms of devices may be utilized in a similar fashion (e.g., AFOs from a given manufacturer or product line, or other types of orthotic devices). The shaded area represents healthy control values from subjects of all ages and genders and this control area (i.e., shaded area) may be adjustable for a more detailed comparison in age and gender.

The system 100 (and the aforementioned RL system 300 or alternative artificial intelligence and/or machine learning paradigms) may also calculate various features from the data received from the IMU sensors 200 so as to give a representative description of the quality of the movement of a given subject. For example, and with respect to gait, one can determine: (1) maximum and minimum acceleration values in both the x-direction (i.e., the axis along the length of the shank) and the z-direction (the axis pointing anteriorly (or posteriorly) to the shank) for the first 10% of the gait cycle; (2) the slope of the trajectory formed for the z-component of the acceleration at push-off (typically between 55-75% of the gait cycle) (see also line 460 for the plot 450 shown in FIG. 4B) with the higher value of the slope representing a better push-off and the unit of the slope being in, for example, m/s²; (3) the maximum angular velocity in degrees/second around the y-direction with the y-axis being defined as the axis going out from the side of the body (lateral for right foot, medial for left foot, with a high value representing higher angular velocity during the swing phase representing a more confident swing from the subject; (4) the rotation span of the 3D rotations of the shank with calibration of the angles being carried out prior to measurement where the subject is asked to stand still for a period of time (e.g., 5 seconds) for the system to record the initial position of the foot; and (6) the summation of the Fourier coefficients based on the quadratic sum of all the acceleration components so as to enable an analysis of the presence of tremors or other instability aspects seen during the gait.

Referring back to FIG. 3B, this gait cycle data is fed into a device/selection Al engine at step 355 in order to determine what type of assistive device(s) and/or supporting gear may be most beneficial to a given user using a training data set from all users (or a subset of users) for the device/selection Al engine. At step 365, the user will receive device selection and/or adaptation recommendations for the selected assistive device(s) and/or supporting gear. The system 100 may then receive further IMU sensor 200 data during use of the selected and/or adapted device at step 375. This IMU sensor 200 data may then be fed into a therapy Al engine at step 385 where various training regimens may be selected using another training data set from all users (or a subset of users) that are using the selected assistive device(s) and/or supporting gear. At step 395, therapy recommendations (e.g., types and intensity of training regimens) are provided by the therapy Al engine in order to maximize the effectiveness of the selected assistive device(s) and/or supporting gear. This process may also be repeated in order to determine upgrades, down-grades or changes to the types of assistive device(s) and/or supporting gear utilized for the treatment of the user.

### Exemplary Lower Body Methodologies—

Users of a given assistive device will typically display an array of gait deviations or abnormal compensatory movements while performing activities of daily living like standing up from the ground, standing up from a chair, walking, stepping over obstacles and the like. The system 100 described in this disclosure detects such abnormalities by means of parameters like acceleration, deceleration, angular velocity, angulation, peak impact and the like. Comparison can be made to different types of normative values. For instance, a comparison in data to what is considered to be normal for an able-bodied individual, or a comparison in data to a peer or comparison in data to the users on prior registered data (e.g., baseline data) may be performed. Based on these identified weaknesses or abnormalities, the system 100 may determine which exercise and what level of difficulty of exercise is best suited for the user to overcome the weakness while also avoiding abnormal compensatory movements. The system 100 will monitor the user during certain reference exercises and provide feedback on the quality of execution. Once that quality has been determined to be sufficient, the difficulty level may be increased (i.e., so-called challenge point management) to further challenge the user to improve on parameters like strength, balance, flexibility, proprioception, and the like. The user's performance in select activities of daily living may be compared against previously registered data to determine progression, and/or regression by the user. In response, further adjustments in the exercise program can be made to achieve optimal user performance.

For example, the system 100 may determine that a user is exhibiting weak push-off at terminal stance while wearing a given ankle foot orthotic or prosthetic leg. Such weak (insufficient) push-off may have significant impact on the level of gait efficiency obtained by the person wearing the device. The level of insufficiency may be defined by the level of anterior tilt of the lower leg at terminal stance as measured by a lower leg sensor, and the peak acceleration which follows the lift-off of the foot off the ground. Below average results on those parameters may indicate poor push off, and therefore suboptimal use of the prosthetic or orthotic device. The cause of such an insufficiency may be associated with multiple factors, including: (1) weak quadriceps muscle of the affected leg; (2) instability on the affected leg; (3) poor hip extension flexibility on the affected leg; (4) poor strength of the hip abductor muscles of the affected leg; and/or (5) lack of proprioception leading to lack of balance.

Each of these individual causes can be addressed with exercises that focus on the improvement of the suspected weakness. Each exercise may also have a minimum expected performance level, meaning that the user of the device is expected to train until such accepted performance level is achieved before the level is cleared and the user of the assistive device is allowed to progress to the next level with a more difficult exercise (i.e., so-called challenge point management). The process of adjusting the difficulty level based on performance in functional assessment as well as the performance during the execution of the exercise is automated and driven by a decision-making process in the artificial intelligence of the system 100. Reassessment of typical activities of daily life including walking, sit-to-stand, incline walking, etc. will provide an insight on the impact of the exercises on the functional performance of the user of the device and the system 100 and log progress or regression with regards to normative data, user data or peer data. Eventually, through persistent exercises the user will start to maximize his/her potential until they eventually graduate from the system. At this time the user can decide to use the system to maintain the higher level of performance, by regularly repeating the advanced level exercises and by performing self-assessment on a regular basis.

FIG. 6A illustrates an exemplary graphical user interface 500 illustrating data collected for an exemplary sit-to-stand exercise. The data collected may include, for example, average adduction/average abduction (e.g., in degrees of movement) as a function of time when the data is being collected. Line 510 may be indicative of correct biomechanics when a user goes from a seated position to a standing position, while line 520 may be indicative of incorrect biomechanics when a user goes from a seated position to a standing position.

FIG. 6B illustrates an exemplary graphical user interface 500 illustrating data collected for an exemplary walking exercise. In this example, three (3) different parameters are being measured. The top plot is of interior/exterior rotation, the middle plot is of flexion/extension, while the bottom plot is of abduction/adduction. Each of these plots illustrates degrees of movement as a function of time. Lines 510 may be indicative of correct biomechanics when a user is performing a normal walking exercise, while line 520 may be indicative of incorrect biomechanics when a user is performing the same walking exercise. Such data may be used as a reference (as discussed supra) to demonstrate progress and/or regression of the user as well as performance while using, for example, different assistive devices or supportive garments.

It will be recognized that while certain aspects of the present disclosure are described in terms of specific design examples, these descriptions are only illustrative of the broader methods of the disclosure and may be modified as required by the particular design. Certain steps may be rendered unnecessary or optional under certain circumstances. Additionally, certain steps or functionality may be added to the disclosed embodiments, or the order of performance of two or more steps permuted. All such variations are considered to be encompassed within the present disclosure described and claimed herein.

While the above detailed description has shown, described, and pointed out novel features of the present disclosure as applied to various embodiments, it will be understood that various omissions, substitutions, and changes in the form and details of the device or process illustrated may be made by those skilled in the art without departing from the principles of the present disclosure. The foregoing description is of the best mode presently contemplated of carrying out the present disclosure. This description is in no way meant to be limiting, but rather should be taken as illustrative of the general principles of the present disclosure. The scope of the present disclosure should be determined with reference to the claims.

1. A method of using an inertial measurement unit (IMU) sensor for collection of biomechanical data, the method comprising:
placing the IMU sensor onto an anatomical portion of a user;
collecting data from the IMU sensor during a prescribed exercise being performed by the user;
processing the data from the IMU sensor using a machine learning or artificial intelligence engine to determine a type of an assistive device for use with the user;
collecting additional data from the IMU sensor when the user is using the assistive device;
processing the additional data using the machine learning or artificial intelligence engine to determine an exercise to be performed by the user when using the assistive device; and
displaying the additional data on a graphical user interface (GUI) along with previously collected data for another performance of the exercise.

2. The method of claim 1, wherein the processing of the additional data using the machine learning or artificial intelligence engine further comprises determining adjustments to the assistive device to further optimize assistive device performance.

3. The method of claim 2, wherein the determining of the adjustments to the assistive device comprises determining dimension adjustments for the assistive device.

4. The method of claim 2, wherein the determining of the adjustments to the assistive device comprises determining a strap configuration for the assistive device.

5. The method of claim 2, wherein the determining of the adjustments to the assistive device comprises determining a wedge type for use with the assistive device.

6. The method of claim 2, wherein the determining of the adjustments to the assistive device comprises determining material types for the assistive device.

7. The method of claim 1, further comprising iteratively collecting additional data from the IMU sensor during additional performances of the exercise performed by the user when using the assistive device and determining a different exercise to be performed by the user using the machine learning or artificial intelligence engine.

8. The method of claim 7, further comprising placing an additional IMU sensor onto the assistive device.

9. The method of claim 8, wherein the iteratively collecting of the additional data comprises iteratively collecting data from both the IMU sensor and the additional IMU sensor.

10. The method of claim 9, wherein the data collected from both IMU sensor and the additional IMU sensor comprises acceleration data and angular velocity data stored in a quaternion number system and the method further comprises:
converting the acceleration data and the angular velocity data from the quaternion number system into three-dimensional (3D) Euler angle data prior to the displaying of the additional data on the GUI.

11. The method of claim 1, wherein the data collected from the IMU sensor comprises acceleration data and angular velocity data stored in a quaternion number system and the method further comprises:
converting the acceleration data and the angular velocity data from the quaternion number system into three-dimensional (3D) Euler angle data prior to the displaying of the additional data on the GUI.

12. The method of claim 11, further comprising sub-dividing the acceleration data and the angular velocity data as a function of gait cycle for the user, wherein the gait cycle is defined as a heel strike by the user followed by an immediately succeeding heel strike by the user.

13. The method of claim 12, wherein the sub-dividing of the acceleration data and the angular velocity data is performed prior to the displaying of the additional data on the GUI.

14. The method of claim 13, wherein the displaying of the previously collected data for another performance of the exercise further comprises displaying data of use of different types of assistive devices during prior performance of the exercise.

15. The method of claim 13, wherein the displaying of the previously collected data for another performance of the exercise further comprises displaying data of use of a different configuration for the assistive device.

16. The method of claim 13, wherein the displaying of the previously collected data for another performance of the exercise further comprises displaying data from other users using the type of the assistive device.

17. The method of claim 13, wherein the displaying of the previously collected data for another performance of the exercise further comprises displaying a shaded area, the shaded area comprising control values collected from other users.

18. The method of claim 13, wherein the displaying of the previously collected data for another performance of the exercise further comprises displaying personal best data for the user of the assistive device.

19. The method of claim 13, further comprising determining a different type of exercise to be performed by the user of the assistive device using the machine learning or artificial intelligence engine.

20. The method of claim 19, further comprising:
collecting additional data from the IMU sensor when the user is using the assistive device and performing the different type of exercise; and
displaying the additional data on the GUI along with previously collected data for another performance of the different type of exercise.
